(19) United States
(12) Patent Application Publication
(10) Pub. No.: US 2020/0069367 A1
(43) Pub. Date: Mar. 5, 2020

(54) SYSTEMS AND METHODS FOR CARDIAC MAPPING AND VECTOR ABLATION WITH A MULTIFUNCTION PATCH ARRAY

(71) Applicant: EPQuant LLC, Haverford, PA (US)

(72) Inventor: Holly Sinnott, Springfield, PA (US)

(21) Appl. No.: 16/558,833

(22) Filed: Sep. 3, 2019

Related U.S. Application Data
(60) Provisional application No. 62/726,269, filed on Sep. 2, 2018.

(51) Int. Cl. A61B 18/14; A61B 5/042

(52) U.S. Cl. CPC A61B 18/1492; A61B 2018/00577; A61B 5/0422

(57) ABSTRACT

The disclosure relates to devices and methods for treatment of cardiac heart rhythm disturbances (arrhythmias). Radiofrequency ablation performed under guidance of a 3-dimensional mapping system has become an important treatment modality for most arrhythmias. Aspects of the disclosure improve the processes for three-dimensional mapping and ablation by improving map stability, precisely directing radiofrequency energy delivery, and predicting radiofrequency ablation lesion formation. Aspects of the disclosure also improve the safety and efficacy of radiofrequency ablation for treatment of heart rhythm disorders.

### CROSS REFERENCE TO RELATED APPLICATIONS

This patent application claims priority to U.S. Provisional Patent Application No. 62/726,269, filed on Sep. 2, 2018, the entire contents of which is incorporated herein by reference.

### FIELD OF THE INVENTION

The present invention relates generally to three-dimensional mapping systems, cardiac arrhythmias, and radiofrequency ablation therapy for those cardiac arrhythmias.

### BACKGROUND OF THE INVENTION

Radiofrequency (RF) ablation is typically applied from a monopolar source at an ablation electrode and a diffusion patch placed on the leg or lower body of a patient. RF ablation when applied this way, if conduction properties of surrounding tissue and fluid is homogeneous, it should result in uniform current delivered to the near field of the monopolar ablation source. The proportion of current delivered to tissue would then be reflective of tissue contact and tissue proximity to the monopolar source.

However, there are a number of assumptions and real world factors involving RF energy delivery that substantially alter current delivery into target ablation tissue. These factors include (1) irregularly shaped ablation electrodes result in irregular amounts of contact between ablation electrodes and tissue and alter local ablation, (2) tissue and fluid have different conduction properties local to the monopolar ablation source resulting in differential current flow into fluid and tissue and alter local ablation (3) actual anatomy has different conduction properties away from the monopolar ablation source may result in differential current flow at the monopolar ablation source and alter local ablation and (4) the dispersion patch is typically placed on a patient's lower extremity or lower body and that dispersion patch placement when combined with realistic anatomy with different conduction properties may result in differential current flow at the monopolar ablation source and alter local ablation.

When the target tissue for ablation is within the vector from the ablation electrode, to the dispersion patch then there may be improved current flow into the target tissue for ablation. This may occur with ablation of the cavo-tricuspid isthmus in which the ablation electrode is superior to the cavo-tricuspid isthmus and the dispersion patch is inferior to the cavo-tricuspid isthmus. This creates a situation where cavo-tricuspid isthmus ablation is improved by the patch placement. In contrast, ablation of the left atrial roof line may be made more difficult by patch placement. In this case, the ablation electrode is inferior to the left atrial roof line and the dispersion patch is also inferior to the left atrial roof line. Thus, the vector from the ablation electrode to the dispersion patch is 180 degree from the target tissue for ablation. This may reduce current flow into the left atrial roof tissue since it is opposite from being directly inline between the ablation electrode and the dispersion patch.

Due to the limitations of the assumptions that are used for standard monopolar ablation electrode with a dispersion patch located on a patient's lower extremity or lower body, RF ablation lesions are difficult to uniformly predict. A simple prediction tool using a specific power and specific contact force may be highly inaccurate in patient specific real world conditions due to the difference in the normal vector of the plane between tissue and blood pool, and the vector between the ablation electrode and the dispersion patch.

### BRIEF SUMMARY OF THE INVENTION

The appended claims define this application. The present disclosure summarizes aspects of the embodiments and should not be used to limit the claims. Other implementations are contemplated in accordance with the techniques described herein, as will be apparent to one having ordinary skill in the art upon examination of the following drawings and detailed description, and these implementations are intended to be within the scope of this application.

In one aspect, the present disclosure includes a novel patch with a magnetic sensor, an electrode element for electrocardiographic and localized impedance measurements (referred to herein as an ECG electrode), and a grounding or dispersive element.

In another aspect, the present disclosure includes a plurality, for example at least six (6), of such patches arranged in a three-dimensional pattern (i.e., an array) substantially surrounding the heart on a patient's chest. In certain embodiments, the so-arranged patches are referenced to a three-dimensional mapping system. In certain embodiments, an electro-anatomic map of the patient's heart is created and referenced to the patch array including measurements of gradients of impedance which are used to improve catheter and map stability. In certain embodiments, cardiac ultrasound may also be used to create a 4-dimensional cardiac map including both cardiac and respiratory motion.

In yet another aspect, the present disclosure involves current delivery from a catheter electrode or electrodes in the patient's heart as a vector to an appropriate multifunction patch or plurality of multifunction patches. In certain embodiments, the current is a radiofrequency current for radiofrequency ablation. In other embodiments, the current is direct current, alternating current, pulsed direct current, or a combination of these current waveforms for irreversible electroporation ablation. In certain embodiments, the vector is calculated by a computer. In certain embodiments, modeling of ablation lesion formation is then performed and applied to the electro-anatomic map as a finite element model of the patient's anatomy. The finite element model may be based on the electro-anatomic map, ultrasound, computed tomography, magnetic resonance imaging, or a combination of these imaging modalities.

### BRIEF DESCRIPTION OF THE DRAWINGS

The summary, as well as the following detailed description, is further understood when read in conjunction with the appended drawings. For the purpose of illustrating the disclosed methods and devices, exemplary embodiments of the methods and devices are shown in the drawings, however, the methods and devices are not limited to the specific embodiments disclosed. In the drawings:

FIG. 1A, FIG. 1B, FIG. 1C, FIG. 1D depict exemplary ablation patch designs

FIG. 2 depicts multifunction patches applied to a patient chest in an array

FIG. 3A and FIG. 3B depict multifunction patches applied to a patient chest in an array with a heart and an ablation catheter.

FIG. 4 depicts an ablation catheter with an ablation vector toward a triangle created by multifunction patches.

FIG. 5 depicts a multi-electrode ablation catheter with ablation vectors through target tissue toward an array of multifunction patches.

FIG. 6 depicts an ablation catheter in different positions with different modes of ablation vectors toward multifunction patches.

FIG. 7 depicts an ablation catheter in different positions with ablation vectors through a target location toward multifunction patches.

### DETAILED DESCRIPTION OF THE INVENTION

While the invention may be embodied in various forms, there are shown in the drawings, and will hereinafter be described, some exemplary and non-limiting embodiments, with the understanding that the present disclosure is to be considered an exemplification of the invention and is not intended to limit the invention to the specific embodiments illustrated. Thus, the following examples are illustrative only and are not a limitation on the present invention.

### I. MULTIFUNCTION PATCH

In one aspect, the present disclosure provides a multifunction patch. The multifunction patch includes a three-dimensional (3D) position sensor and a dispersion element. In certain embodiments, the multifunction patch also includes an ECG electrode.

In certain embodiments, the 3D position sensor includes one or more magnetic field sensors. In some such embodiments, the one or more magnetic field sensors create a signal in response to a magnetic field emitted by a magnetic field emitter (e.g., a locator pad that is placed beneath the patient during the procedure). In some embodiments, the 3D position sensor includes a tri-axial sensor, preferably a tri-axial sensor that includes three orthogonally configured coils.

In other embodiments, the multifunction patch is located using an alternate method such as a fluoroscopic marker wherein the fluoroscopy system is capable of registering the 3D location to the patient's anatomy and/or a 3D mapping system. Other alternate methods are to use cameras and stereoscopic computer vision to locate the multifunction patch and register the 3D location of the patch to the patient's anatomy and/or a 3D mapping system.

In certain embodiments, the dispersion element is an electrode. In some such embodiments, the dispersion element comprises a conductive wire that is electrically connected to a conductive gel. The conductive gel is exposed so that it may be placed in contact with a patient's skin. In some such embodiments, the dispersion element (e.g., electrode), or a portion thereof, such as the conductive gel, is electrically isolated from other elements of the multifunction patch. For example, the dispersion element may be surrounded by an adhesive material, which additionally serves to fix the electrode to a patient's skin. An exemplary conductive gel comprises a gel substance and an electrolyte. An example of the electrolyte is Ag⁺/Ag⁺Cl⁻.

In certain embodiments, the ECG electrode comprises a conductive wire that is electrically connected to a conductive gel. The ECG electrode, or a portion thereof, such as the conductive gel, is surrounded by an electrically insulating (and, optionally, adhesive) material. As described herein, a conductive gel may comprise a gel substance and an electrolyte.

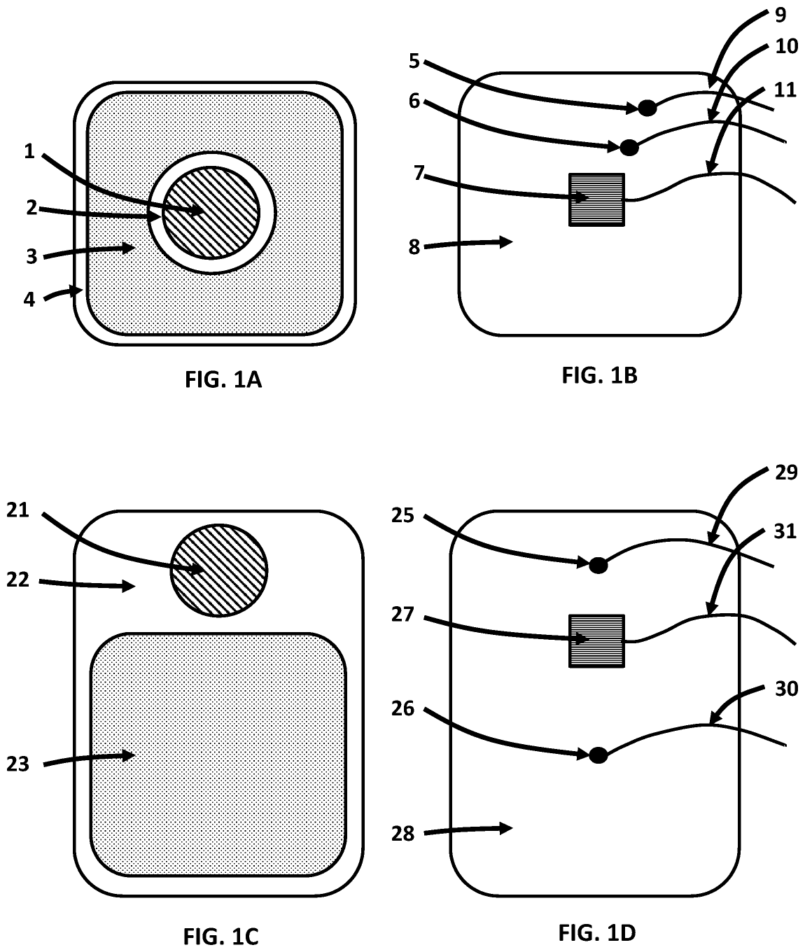

FIG. 1A shows the side of the multifunction patch the side that would be placed on the human body. The thatched area depicts the region of the ECG electrode 1. The dotted is gray area depicts the region of the dispersion element 3. Between these regions and around the outside is an adhesive material 2, such as foam tape, used to affix the patch to the human body. FIG. 1B depicts the other side of the multi-function patch with 3 cables 9, 10, 11 which provide connections to (1) the magnetic sensor, (2) the ECG electrode, and (3) the dispersion element.

As shown in FIG. 1A, the ECG electrode 1 and dispersion element 3 may be electrically isolated from other elements of the multifunction patch. For example, an electrical isolation element 2 (e.g., comprising, for example, an adhesive insulating material, which can be used to fix the electrode to a patient's skin) and, thereby, isolate certain components of the patch from other components. A second electrical isolation element 4 surrounds the dispersion element 3 for additional electrical isolation. The opposite side of the patch (FIG. 1B) includes an insulator material such as foam insulation 8. As shown in FIGS. 1A and 1B, cables 9 and 10 connect to electrical connectors 5 and 6 respectively to form an electrical connection to the electrodes 1 and 3 respectively. Cable 11 connects to the 3D position sensor 7 and is near the center of the multifunction patch. Cables 9, 10, 11 may be configured for connection to a processor (not shown) such that a signal (e.g., an electrical signal) generated by, for example, position sensor 7 can be transmitted to the processor. The processor may be, for example, configured to determine the position coordinates of the patch. In an alternate embodiment, a signal generated by position sensor 7 is wirelessly transmitted to the processor; thus, the patch may be configured to wirelessly communicate via Bluetooth, Wi-Fi, Near Field Communication (NFC), Ultra-Wide Band (UWB), and/or any other short-range and/or local wireless communication protocol (e.g., IEEE 802.11 a/b/g/n/ac).

The exemplary design shown in FIG. 1A has an electrical isolation element 2 between the ECG electrode 1 and the dispersion element 3. Alternative configurations may include a single electrode that is used as both an ECG electrode and a dispersion element.

An alternative configuration with a single electrical isolation element 22 that surrounds the both the ECG electrode 21 and the dispersion element 23 is depicted in FIG. 1C. The opposite side of the patch (FIG. 1D) includes an insulator material such as foam insulation 28. As shown in FIGS. 1C and 1D, cables 29 and 30 connect to electrical connectors 25 and 26 respectively to form an electrical connection to the electrodes 21 and 23 respectively. Cable 31 connects to the 3D position sensor 27. Cables 29, 30, 31 may be configured for connection to a processor (not shown) such that a signal (e.g., an electrical signal) generated by, for example, position sensor 27 can be transmitted to the processor. The processor may be, for example, configured to determine the position coordinates of the patch. In an alternate embodiment, a signal generated by position sensor 27 is wirelessly transmitted to the processor; thus, the patch may be configured to wirelessly communicate via Bluetooth, Wi-Fi, Near Field Communication (NFC), Ultra-Wide Band (UWB), and/or any other short-range and/or local wireless communication protocol (e.g., IEEE 802.11 a/b/g/n/ac).

In one aspect, the present disclosure provides a multifunction patch having a 3D position sensor, a first electrically isolated electrode element, and a second electrically isolated electrode element. In certain embodiments, the first electrically isolated electrode element is intended to be used for impedance and electrocardiogram measurements. In certain embodiments, the second electrically isolated electrode element is intended to be used as a dispersion or grounding element for RF energy delivery between an ablation electrode and that second element and also for impedance measurements. Having two separate electrode elements minimizes ECG noise created by RF energy delivery.

In one aspect, the present disclosure provides a 3D position sensor, a small electrode, and a large electrode. In certain embodiments, the 3D position sensor is a magnetic sensor. In certain embodiments, the small electrode enables ECG measurement and localized impedance measurement. In certain embodiments, the large electrode is a dispersion electrode, which also allows for impedance measurement.

### II. ARRAYS OF MULTIFUNCTION PATCHES

In one aspect, the present disclosure provides a plurality of multifunction patches as described herein. In certain embodiments, each multi-function patch has (1) a magnetic sensor for defining/detecting the patch position in 3-dimensional space, (2) an ECG electrode for measuring and electrograms (and, additionally, the impedance between the ECG electrode and other electrodes within the human body or on the surface of the body), and (3) a dispersion element for delivering current between an ablation catheter and the dispersion patch.

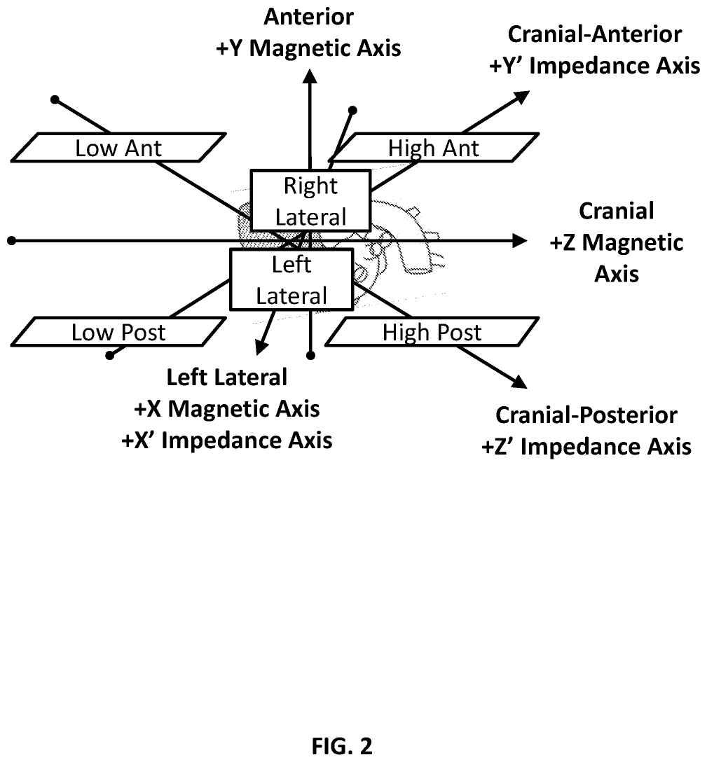

In another aspect, an array of multifunction patches are arranged on the human body to substantially surround the heart. For example, FIG. 2 depicts an exemplary array comprising at least six (6) multifunction patches labeled as (1) high anterior, (2) low anterior, (3) high posterior, (4) low posterior, (5) left lateral, and (6) right lateral. In this particular example, the high anterior multifunction patch is placed in the midclavicular line, left pectoral region, inferior to the left clavicle; the low anterior multifunction patch is placed in the midclavicular line, at the junction of the rib cage and the abdomen; the high posterior multifunction patch is placed directly posterior to the high anterior multifunction patch; the low posterior multifunction patch is placed directly posterior to the low anterior multifunction patch; the left lateral multifunction patch is placed directly lateral to the approximate location of the heart in the left midaxillary line; and the right lateral multifunction patch is placed directly opposite the left lateral multifunction patch in the right midaxillary line.

In certain embodiments, the multifunction patches are arranged in a way that approximates an octahedron or square bipyramid. In some such embodiments, the vertices of the pyramid are located left lateral and right lateral to the heart on the chest wall and the 4 corners of the base of both pyramids are located anterior-cranial, anterior-caudal, posterior-cranial, and posterior-caudal to the heart on the chest wall. Thus, the heart and is substantially surrounded by multifunction patches. Moreover, in this particular example, since the multifunction patches are located relatively close to the heart, current flow from the ablation electrode locally will be modified by the location of the multifunction patch.

In certain embodiments, an alternative configuration to the six multifunction patch configuration is employed. For example, a seventh multifunction patch may be placed at intersection of the coronal plane and the left midclavicular line. In some such embodiments, the seven multifunction patch system has five multifunction patches in left midcalvicular sagittal plane and form an irregular pentagon; the right lateral and left lateral multifunction patches are then the vertices of the pyramids to form a pentagonal bipyramid.

In certain embodiments, the array of multifunction patches comprises 8 or more patches, such as 8 to 40 patches. In some such embodiments, the patches are dispersed on the torso of the patient. The locations of the patches are calculated by the location system. In certain embodiments, a Delaunay triangulation may be performed on the patch locations to construct a triangulated surface using the patches as vertices. Thus, any set of multifunction electrode patches may be used to as the multifunction patch configuration.

In certain embodiments, an array of dispersion electrode patches are placed on a patient's body. The array may comprise six or more, alternatively eight or more, such as 8 to 40, dispersion electrode patches. In some such embodiments, the patches are dispersed on the torso of the patient. The locations of a first set patches are used to define the relationship between impedance measurements between the patches and a location system. Impedance measurements are made between the first set of patches and the second set of patches to identify the locations of the second set of patches. Thus, dispersion electrode patches may be used without a location sensor to generate an array of dispersion electrode patches on the torso of the patient that substantially surrounds the heart. The next paragraph describes this method in more detail. In certain embodiments, a Delaunay triangulation may be performed on the patch locations to construct a triangulated surface using the patches as vertices. Thus, any set of dispersion electrode patches may be used to as the patch configuration when it is normalized to a set of patches with location sensors.

Those of skill of the art will recognize an alternative method to using a multifunction patch to substantially surround the heart and perform vector ablation. In an alternative embodiment of the present invention, the following method may be used to determine the location of an array of dispersion electrode patches. Those dispersion electrode patches may substantially surround the heart and be used to modify the current density leaving the ablation electrode. A first set of electrodes associated with a 3D positioning method are placed on a patient. The location of each of the electrodes is determined in 3D space. One method for determining the 3D positions is using a magnetic positioning system with locations calculated accurately in 3D space. The physical distance between each of the first set of electrodes is calculated. Each of the first set of electrodes may be considered a vertex of a connected graph. Each edge of the connected graph is labeled by the physical distance. A plurality of impedance measurements made between first set of electrodes wherein each electrode has an impedance measurement made relative to each of the other electrodes. Each of the first set of electrodes is a vertex on the connected graph and each of the edges is labeled with a physical distance. The plurality of impedance measurements is added to the connected graph edges so that each edge is labeled with both a physical distance and an “impedance distance” wherein the impedance distance is the impedance between the electrodes. A second set of electrodes may be placed on a patient. The second set of electrodes may act as dispersion electrodes for ablation of cardiac tissue. If the second set of electrodes does not have an associated 3D positioning method such as a magnetic sensor, then the location of the second set of electrode will need to be calculated. A plurality of impedance measurements can be made between each of the second set of electrodes to each of the first set of electrodes. Now the impedance distance is known between each of the second set of electrode and each of the first set of electrodes. The second set of electrodes may now be added to the connected graph. Based on the impedance distances in the connected graph, the position of each of the second set of electrodes may be estimated relative to the first set of electrodes. Since each edge of the connected graph between the first set electrodes contains both a physical distance and an impedance distance, and the impedance distance can be measured between each of the second set of electrodes and the first set of electrodes, now a physical distance may be estimated between the first set of electrodes and a second set of electrodes. A variety of computational methods may be chosen to perform this task. For example, an estimate of the location may be made, an error estimate calculated, and the location updated to minimize error in an iterative fashion until the error is adequately minimized. The result is the approximate 3D position of each of the second set of electrodes relative to the first set of electrodes. Although the second set of electrodes does not include a position sensor, the location of the second set of electrodes is calculated based on measurements from the first set of electrodes and now that second set of electrodes may be used within the 3D location system for dispersion electrodes to perform ablation.

A patch that has a computed location may functionally be used as a multifunction patch since the function of location is performed via measurements from the patch and computational software. For the purposes of the description, when a multifunction patch is described in this text, a multifunction patch may be a patch with any of these combinations: (1) a location sensor element (e.g., a 3D position sensor) and a dispersion electrode; (2) a location sensor element (e.g., a 3D position sensor), a dispersion electrode, and an ECG electrode element; (3) a computed location and a dispersion electrode; (4) a computed location, a dispersion electrode, and an ECG electrode element.

### III. ABLATION METHODS

In another aspect, the present disclosure provides a method for performing a tissue ablation procedure.

In certain embodiments, the tissue ablation procedure is radiofrequency (RF) tissue ablation, and more particularly RF cardiac tissue ablation. Radiofrequency ablation is performed using an ablation catheter, a radiofrequency (RF) generator, and a multifunction patch, or preferably a plurality of multifunction patches described herein. The circuit for ablation consists of (1) RF current produced by the RF generator, (2) a cable connected from the RF generator to an ablation catheter electrode, (3) current flow from the ablation catheter electrode through the patient's body to one or more multifunction patches placed on the patient's skin, and (4) one or more cables connecting the one or more multifunction patches back to the RF generator. Resistance to RF current within the patient's body to RF current produces heat. The location of highest RF current density is near the ablation catheter electrode. RF current delivery causes resistive heating and generates an adequate temperature rise in the tissue to denature proteins and result is cell death. This produces an RF ablation lesion that may be used to treat a cardiac arrhythmia

Alternatively, the tissue ablation is electroporation ablation. Irreversible electroporation is an alternative type of ablation to traditional radiofrequency ablation. Irreversible electroporation involves a cardiac ablation catheter with an electrode or array of electrodes, a dispersion electrode on a patient's skin, and delivery of electrical current. The types of electrical current may be direct current, alternating current, pulsed direct current, or a combination of these current waveforms. The local electric field generated by the electrical current for electroporation has the same constraints as the electrical current for RF current delivery. While the present disclosure generally refers to RF ablation, irreversible electroporation may also benefit from use of vector ablation to improve delivery of electrical current into tissue targeted for ablation.

In certain embodiments, the method comprises placing a plurality of locatable dispersion patches on the patient. In some such embodiments, each of the plurality of locatable dispersion patches is locatable by a mapping system. In some such embodiments, each of the plurality of locatable dispersion patches comprises a 3D position sensor. For example, each of the patches of the may include a magnetic sensor, and each of the magnetic sensors can be tracked by a three-dimensional mapping system. In some such embodiments, the three-dimensional mapping system is referenced to the fluoroscopy table or other table that the patient rests on. In certain embodiments, the locatable dispersion patch is a multifunction patch disclosed herein (e.g., having at least a 3D position sensor and a dispersion element).

The method may further comprise advancing a catheter via the arterial or venous system to the heart. In some such embodiments, that catheter comprises a plurality of electrodes and a magnetic sensor. In certain embodiments, more than one catheter may be positioned in the heart and each catheter may have magnetic sensor and electrodes.

In certain embodiments, the multifunction patches placed on the patient's body serve as 3D points that are tracked by a 3D location are 3D points. In some such embodiments, those 3D points are used to form triangles of a polyhedron (e.g., those 3D points are vertices joined by edges). In the exemplary case of 6 multifunction patches, there are 8 triangles generated by the 6 3D points or vertices. In certain embodiments, the ablation electrode is within the volume of the polyhedron and is another 3D point. An ablation vector may be selected manually or calculated by a computer.

Multiple modes for determining the ablation vector may be utilized. Ablation procedures include a variety of targets. Multiple modes for determining the ablation vector will be described to accommodate these varied ablation targets. There are a variety of factors during an ablation procedure that alter how an ablation is performed. At times, there is a focal location that is the target for ablation. At times, a linear lesion along a surface of the heart is the target for ablation. At other times, homogenization of a large area of scar is the target for ablation and large patch of tissue is the target for ablation. Often there are structures identified that an operator wishes to avoid in an ablation. These structures may include the esophagus, the phrenic nerve, coronary arteries, the sinus node, the AV node, the HIS bundle. Multiple modes of determining the ablation vector may be used to accomplish the task of ablation of tissue, and also avoiding tissues desired not to be ablated. Described herein are exemplary modes of calculating ablation vectors and these modes may be used in combination with one another: (1) manually selected ablation vector, (2) 3D model surface normal ablation vector, (3) ablation vector aligned to force vector, (4) ablation vector calculated to transect a specific ablation target, (5) ablation vector blanking regions wherein structures identified to be avoided are excluded from being included in the ablation vector.

In certain embodiments of the methods disclosed herein, an ablation vector is manually selected using and software interface in the 3D mapping or 3D location system. The manually selected ablation vector may be very useful in areas where the anatomy is not well defined by the mapping system. Tactile feedback from manual catheter motion and operator experience with complex anatomy and arrhythmias may lead to a perception that a deeper ablation lesion is required in a specific location in the anatomy. An operator may use the manually selected vector for this portion of the ablation. Also, for performing a linear ablation lesion, it may be desirable to have a consistent ablation vector throughout the duration of application of the linear ablation lesion. A manually selected vector may be ideal for this application.

In certain embodiments of the methods disclosed herein, a surface normal vector is calculated for the tissue in the region of interest. That surface normal may be a surface normal to the 3D electro-anatomic map from the 3D mapping system. That surface normal vector is the ablation vector. The multifunction patch that best aligns to the surface normal vector, or preferably two or more multifunction patches that align to the surface normal vector, is/are identified. Ablation is performed by delivering a current between the ablation electrode and the multifunction patches. The quantity of current that is delivered to each of the multifunction patches is calculated to best approximate the desired ablation vector. The current delivered may be designed for radiofrequency ablation or for electroporation ablation. As the ablation is performed and the ablation catheter is moved, the surface normal to that location may be recalculated and the ablation vector updated to be appropriate to the new location.

In certain embodiments of the methods disclosed herein, an ablation vector is determined by the catheter contact force vector. Some modern ablation catheters have the ability to measure contact force. The measured contact force may include a calculated vector of that contact force. In this mode, the ablation vector is assigned to be the same as the contact force vector. As the contact force vector is updated, data from the contact force vector may be used to update the ablation vector as an automated feature.

In certain embodiments of the methods disclosed herein, an ablation vector may be calculated to transect a specific ablation target. The ablation target may be manually selected or computed from the results of a 3D mapping system. Often, it is difficult to maintain catheter stability in a specific location when targeting tissue for ablation. In this mode, the ablation vector is calculated and updated on a regular basis so that if the ablation catheter moves during ablation, the ablation vector is updated to continue to transect the ablation target. Thus, a greater amount of current may be delivered to a specific ablation target despite catheter motion or catheter slipping along a surface of the heart.

In certain embodiments of the methods disclosed herein, anatomic structures are identified to be avoided during an ablation procedure. Since the location of structures to be avoided are identified in 3D space in a 3D mapping system, and the ablation catheter electrode and multifunction patches are also known in the 3D mapping system, exclusion vectors may be calculated to avoid or minimize delivering current into those structures. The calculated exclusion vectors may work as part of another mode for determining an ablation vector.

In preceding sections there were descriptions on how to (1) locate multifunction patches, (2) create a triangulated mesh or polyhedral from the multifunction patches, (3) select an ablation vector from the ablation electrode to the multifunction patches. The next step is to apply a current or currents between the ablation electrode or electrodes and the multifunction patches to preform ablation. Types of ablation using currents include RF ablation and electroporation. Application of current or currents to multifunction patches may be performed in a number of ways. On the least complex side, a single current may be applied between the ablation electrode and a single multifunction patch. A more complex approach is to identify two or more multifunction patches that are oriented in the direction of the ablation vector and apply the current between the ablation electrode and two or more multifunction patches. A higher fidelity approach is to identify a the triangle of the polyhedra that the ablation vector transects and independently apply currents between the ablation electrode and each of the multifunction patches that comprise the triangle vertices in a calculated manner. Ablation catheters may have more than one ablation electrode. Currents may be applied between each of the ablation electrodes and one or more of the multifunction patches to shape the desired application of current for an optimal ablation.

In certain embodiments, an ablation vector is generated and a vector from the ablation electrode to each of the multifunction patches is generated (“dispersion vectors”). The angles between the ablation vector and the dispersion vectors are calculated. The dispersion vector with the minimum angle to the ablation vector is selected as the single best dispersion patch. Then current is applied for ablation between the ablation electrode and the single best dispersion patch. This is the simplest method to perform vector ablation. However, it also has the largest differences between angles since when there is a 6 patch system, there is approximately 90 degrees between each patch and its nearest neighbor patches. This approach is the simplest to implement and will have a large benefit compared to conventional ablation systems that utilize a single dispersion patch on a lower limb or lower portion of the body.

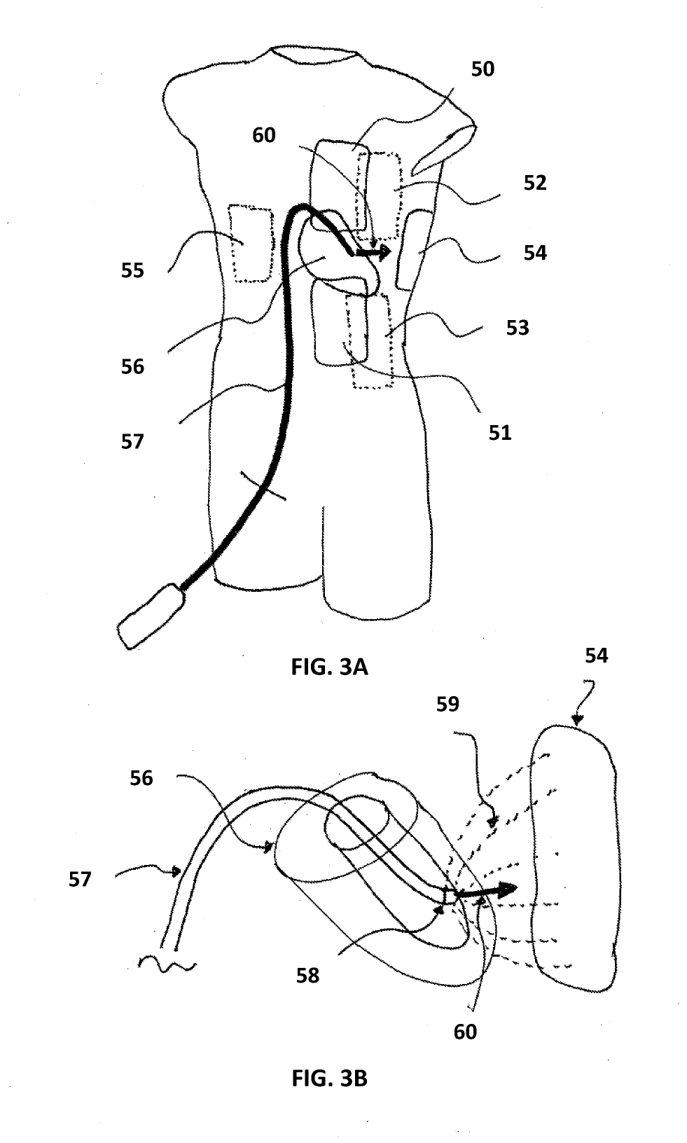

A visualization of the relationship between the ablation catheter and ablation electrode, array of multifunction patches, ablation vector, and ablation current relative to the heart and body of a patient is depicted in FIG. 3A and FIG. 3B. FIG. 3B is a close in view of FIG. 3A and depict the left ventricle or heart 56, with ablation catheter 57 and ablation electrode 58 with left lateral multifunction patch 54. In FIG. 3A, the torso of a patient is depicted with an ablation catheter 57 with its distal ablation electrode in the heart 56. The heart is surrounded by an array of multifunction patches. In this example, there are 6 multifunction patches in an arrangement similar as FIG. 2. Those multifunction patches are high anterior 50, low anterior 51, high posterior 52, low posterior 53, left lateral 54, and right lateral 55. The desired ablation vector 60 points left lateral relative to the ablation electrode 58. In this example, left lateral multifunction patch 54 is in the direction of the ablation vector 60. Thus, when current 59 is applied between the ablation electrode 58 and the dispersion electrode of multifunction patch 54, the shape of the current field is modified by the spatial relationship between the ablation electrode and the dispersion electrode to increase the current delivered into the heart tissue along the path of the desired ablation vector 60.

In certain embodiments, an ablation vector is generated and a vector from the ablation electrode to each of the multifunction patches is generated (“dispersion vectors”). The angles between the ablation vector and the dispersion vectors are calculated. The dispersion vectors with angles less than a threshold to the ablation vector are selected. Thus, multiple patches may be selected to be included in the ablation circuit. Current may then be applied. Methods for current application include delivery of current between the ablation electrode and all of the selected dispersion patches. Methods also include delivery of current from the ablation electrode to each of the selected dispersion patches with an independent circuit to differentially impact current flow. Thus, more current may be applied to the patch with least angle between the ablation vector and the dispersion vector and less current delivered to the patch(es) with greater angles between the ablation vector and the dispersion vector(s). This approach is more complex than the simplest approach and offers some greater refinement of the current delivery away from the ablation electrode and may improve the ablation outcome.

In certain embodiments, an ablation vector is generated and projected to the triangulated mesh of multifunction patches. The multifunction patches that comprise the triangle that is transected and location of the transection is identified and appropriate currents a calculated and applied between each of the multifunction patches and the ablation electrode. This approach is more complex, but offers the greatest refinement in matching the current delivery to the desired ablation vector. For example, the following is exemplary of one such method. A 3D map of the heart is generated as a first step using a 3D mapping system. Based on the 3D map of the heart and the arrhythmia mechanism, a location for ablation is selected. The ablation electrode is moved to the location targeted for ablation. The surface normal to the location targeted for ablation is calculated from the 3D mapping system. The surface normal in this method is also the intended ablation vector. The ablation vector from the ablation electrode is then projected to the polyhedron formed from the multifunction patches. The point that the ablation vector intersects with a triangle of the polyhedron is identified as point ‘D’. The three multifunction patches that serve as the vertices of the triangle are identified. Current is applied between each of those three multifunction patches to the ablation electrode.

Current is calculated to approximate the ablation vector. The current generator independently applies current between the ablation electrode and each of the multifunction patches. In certain embodiments, the current generator comprises multiple generators functioning in parallel with each channel associated with a multifunction patch. Thus, the current between each multifunction patch and the ablation electrode may be independently controlled. The total current is the sum of each of the currents delivered to the multifunction patches.

Multiple methods may be used to calculate the current to be applied between the ablation electrode and each of the multifunction patches. The following is exemplary of one such method for calculating the current to be applied to each of multifunction patches. Multifunction patches A, B, and C are associated with vertices A, B, and C. The point that the ablation vector intersects with a triangle of the polyhedron is identified as point ‘D’. The distances AD, BD, and CD are calculated using the Pythagorean theorem. A fraction of the total current (I_{Total}) will go to each of the multifunction patches and that fraction will be inversely proportional to the distance between the vertex and point ‘D’. The fraction of current to multifunction patch A is “I_{A}”, multifunction patch B is “I_{B}”, and multifunction patch C is “I_{C}”. The current for each of the multifunction patches may be calculated as follows:

I_{A}=I_{Total}*(1/AD)/(1/AD+1/BD+1/CD)

I_{B}=I_{Total}*(1/BD)/(1/AD+1/BD+1/CD)

I_{C}=I_{Total}*(1/CD)/(1/AD+1/BD+1/CD)

For further illustration, a common ablation power is 40 Watts with an impedance of 110 Ohms. Current is the square root of power over resistance and in this example calculates to 0.603 Amps. If the distance AD is 2 cm, BD is 10 cm, and CD is 20, then application of the above formulas calculate I_{A} as 0.463846 Amps, I_{B} as 0.092769 Amps, and I_{C} as 0.46385 Amps for a total power of 0.603 Amps. The sum of the three current vectors approximates the desired ablation vector. One skilled as the art will recognize that the illustrated method above is exemplary and that multiple solutions exist for calculation of currents to be applied between the multifunction patch and the ablation electrode.

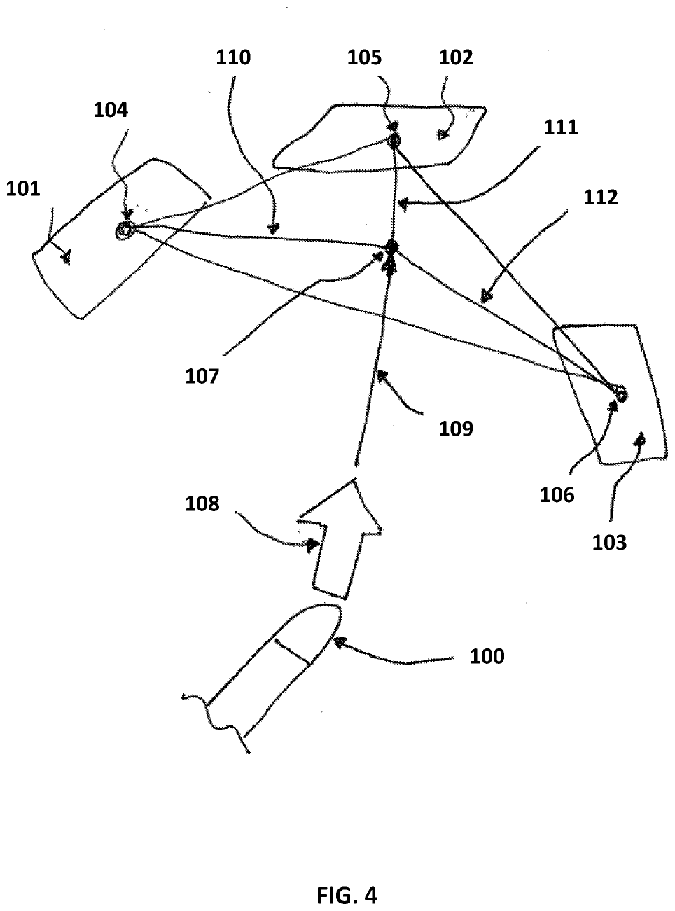

This method is further illustrated with FIG. 4 with ablation electrode 100 and multifunction patches 101, 102, 103. For each multifunction patch 101, 102, 103, the 3D location of the dispersion electrode is calculated as 104, 105, 106 respectively and become vertices of a triangle. In this illustration, only one triangle of the plurality of triangles substantially surrounding the ablation electrode is shown of the triangulated mesh and only three of the plurality of multifunction patches is shown. In this illustration, ablation electrode 100 has an associated ablation vector 108 selected. That ablation vector is projected along path 109 to point 107 (point ‘B’ in above calculations) on the surface of the triangle defined by vertices 104, 105, 106 (points ‘A’, ‘B’, ‘C’ in above calculations). From those points, the segments 110, 111, 112 (lines ‘AD’, ‘BD’, ‘CD’ in above calculations) are identified. Using the above calculation method, appropriate ablation currents may be calculated and then applied between the ablation electrode and the multifunction patches to deliver maximal current along the desired ablation vector.

The vector is calculated using current. In the case of RF energy, it is current that travels through a resistor to generate heat. This is known as resistive heating. Heat that travels to additional tissue from the resistive heating is known as conductive heating. In the case of current used for electroporation, the lesion formation is proportional to current delivered. In both cases, it is current delivery into tissue that is critical for ablation lesion formation. Power is dependent on resistance. Since resistance depends on the cumulative resistance of tissues between the ablation electrode and the dispersion electrode on the multifunction patch and that resistance may be variable depending on location of the dispersion patch, it is not desirable to calculate the vector based on power alone. Thus, current is the preferred unit for calculating the ablation vector and energy delivery.

When a plurality of multifunction patches are arranged in a array to substantially surround the heart idealized assumptions about uniform distribution of current flow out of a uniform ablation electrode do not hold true because the multifunction patches are now positioned closer to the heart. Moreover, this approach allows for current to be directed from the ablation electrode differentially toward one or more of the multifunction patches. For example, current may be directed to only one of the plurality of multifunction patches. Thus, the term “differentially directing” encompasses directing current to only one of a plurality of multifunction patches as well as directing different (or the same) amount of current to two or more, alternatively three or more, of the plurality of multifunction patches. By having a plurality of multifunction patches which are located as an array surrounding the heart, appropriate multifunction patch or patches may be selected so that the desired tissue for ablation is located between the ablation electrode and the multifunction patch. This will enhance current flow into the tissue targeted for ablation and improve ablation efficacy.

Three dimensional cardiac mapping systems are commonly used with the diagnostic catheters and ablation catheters to create an electro-anatomic map of the arrhythmia. An electro-anatomic map is a three-dimensional representation of geometry of the heart labeled with colors that representing timing of local activation during an arrhythmia. Analysis of the electro-anatomic map is used to identify mechanisms of an arrhythmia and critical portions of the circuit that may be targeted for ablation.

An exemplary three dimensional cardiac mapping system uses three or more coils mounted in a “locator pad” that is attached underneath the fluoroscopy table where a patient lies for the procedure. Those coils emit magnetic fields that are used to triangulate the location of magnetic sensors as a three-dimensional location technology. As described herein, a multifunction patch having one or more magnetic sensors can be tracked by such mapping systems. Moreover, such multifunction patches can be used to track the location of the patient relative to the coils. In certain embodiments, a computer is used to calculate and track the location of the magnetic sensors relative to the coils. In certain embodiments, electrograms are acquired from the catheter electrodes simultaneous to the positional data. The computer may then use that data to produce an electro-anatomic map.

In operation, a 3D mapping system is used to generate a model of the cardiac structure of interest. A region of interest for ablation is identified based on the 3D map and additional electrophysiologic observations in the case. The ablation catheter is advanced to the region of interest for ablation. The position of the catheter ablation electrode relative to the surface of the tissue of interest is identified.

A current problem in radiofrequency ablation is the vector of radiofrequency energy between the ablation catheter electrode and a dispersion element. A fundamental assumption when instrumentation is performed with a dispersion patch on a lower extremity or lower part of the body is that the distance between the ablation electrode and the dispersion element will minimize potential dispersion patch-ablation electrode anatomic interactions. More specifically, when the distance is large, current is expected to leave the ablation electrode relatively uniformly and be capable of equal ablation in all directions. That assumption is not correct in real world conditions. Furthermore, when that assumption is not correct it makes affective radiofrequency ablation of cardiac tissue difficult and may increase the risk of procedural complications while reducing procedural efficacy.

Thus, as described herein, “vector ablation” seeks to overcome the limitations of traditional radiofrequency ablation by manipulating the location of the dispersion patch according to the needs of ablation. Specifically, dispersion patches are placed in an array around the heart such that the vector of the dispersion patch may be selected manually based on the 3-dimensional location of the ablation tissue, or the dispersion patch may be selected by a computer to determine an optimal vector of ablation. Importantly, the optimal radiofrequency ablation vector may be a combination of dispersion patches such that the optimal radiofrequency energy delivery is calculated as a first proportion of RF energy dispersed at first patch, a second proportion of RF energy dispersed at a second patch, and a third proportion of energy dispersed at a third patch. The locations of the dispersion patches are placed in a geometric pattern around the heart. In an alternative embodiment, dispersion patches may be placed in a different geometric pattern around the heart. The geometric pattern of dispersion patches generates a pattern of triangles around the ablation catheter electrode. The dispersion patches may be considered nodes or corners of those triangles. Any ablation vector that is desired may be calculated or selected away from the ablation catheter electrode and the appropriate combination of dispersion patches and relative energy to each of those dispersion patches may be selected to derive that ablation vector. Since the system senses the location of the dispersion patches relative to the ablation electrode, additional radiofrequency energy may be delivered into cardiac tissue which is selected by three-dimensional mapping and entrainment maneuvers as important to cardiac arrhythmias. Vector ablation may result in a higher proportion of energy delivered into cardiac tissue and thereby may be more efficacious than traditional radiofrequency energy ablation. Vector ablation may result in deeper penetration of resistive and conductive heating resulting in ablation of tissue that may not be ablated by traditional radiofrequency ablation.

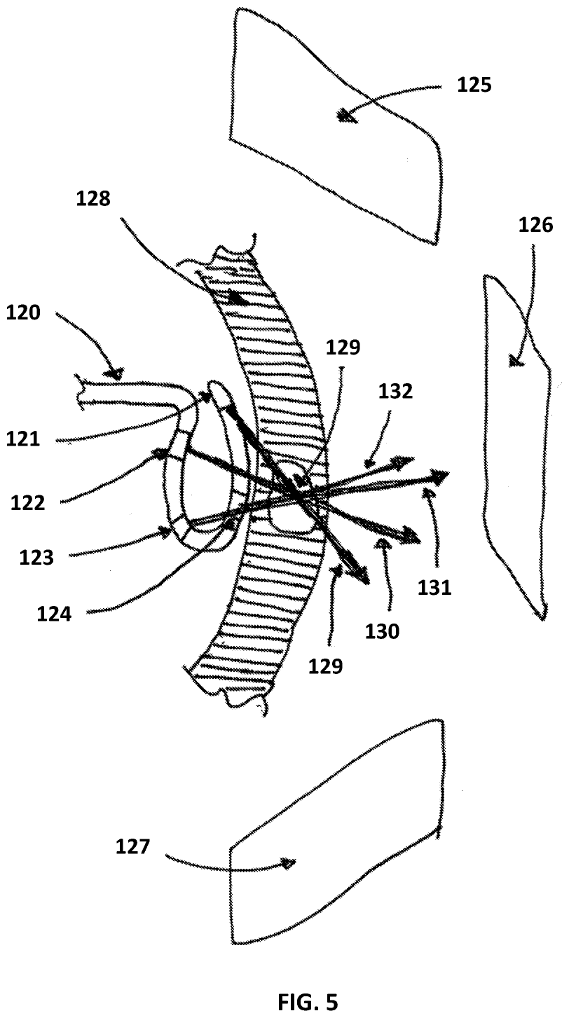

In certain embodiments, radiofrequency ablation energy may be delivered from a single electrode at an ablation electrode. Radiofrequency ablation energy may also be delivered from a plurality of electrodes at an ablation electrode or an ablation catheter including the distal shaft portion of the ablation catheter. The vector of ablation may be further manipulated by generating overlapping currents from the electrodes on the ablation catheter and the dispersion patches. FIG. 5 illustrates tissue 128 with ablation target tissue 129 that is between the distal aspect of ablation catheter 120 and multifunction patches 125, 126, and 127. The distal ablation catheter 120 has ablation electrodes 121, 122, 123 and 124 associated with ablation vectors 129, 130, 131, 132 respectively that pass through target tissue 129 toward an array of multifunction patches that include dispersion electrodes. Appropriate ablative currents may be calculated for each ablation electrode to each of the dispersion electrodes to result in ablation current delivery aligned to each ablation vector.

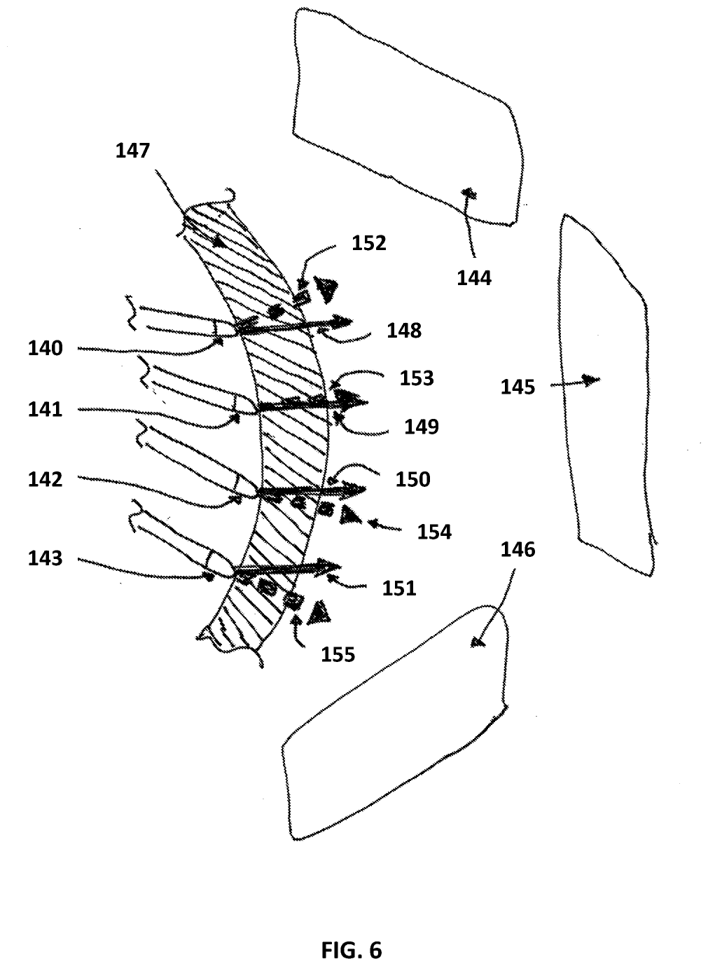

In certain embodiments, ablation may be delivered as a series of ablations to tissue. One mode for calculating the ablation vector is manual selection of a specific vector using inputs to a computer system. One alternative mode for calculating the ablation vector is the vector that is the normal to the surface of the tissue. Other alternative modes for calculating the ablation vector are discussed in the text. FIG. 6 illustrates creating of a series lesions in tissue 147. In this illustration, the distal aspect of an ablation catheter is moved in sequential positions with the ablation electrode initially in position 140 then sequentially moved to positions 141, 142, and 143. Ablation vectors may be manually selected 148, 149, 150, 151 (solid line vectors) or may be the normal to the surface of the tissue 152, 153, 154, 155 (dashed line vectors). The illustrated series of multifunction patches 144, 145, 146 are part of a complete set of multifunction patches that substantially surround the potential ablation electrode positions. The complete set of multifunction patch locations may be used as vertices to create a triangulated mesh. Ablation vectors 148, 149, 150, 151 (manual mode) or 152, 153, 154, 155 (surface normal mode) may be projected to the triangulated mesh and appropriate ablation currents delivered between the ablation electrode and the multifunction patch array.

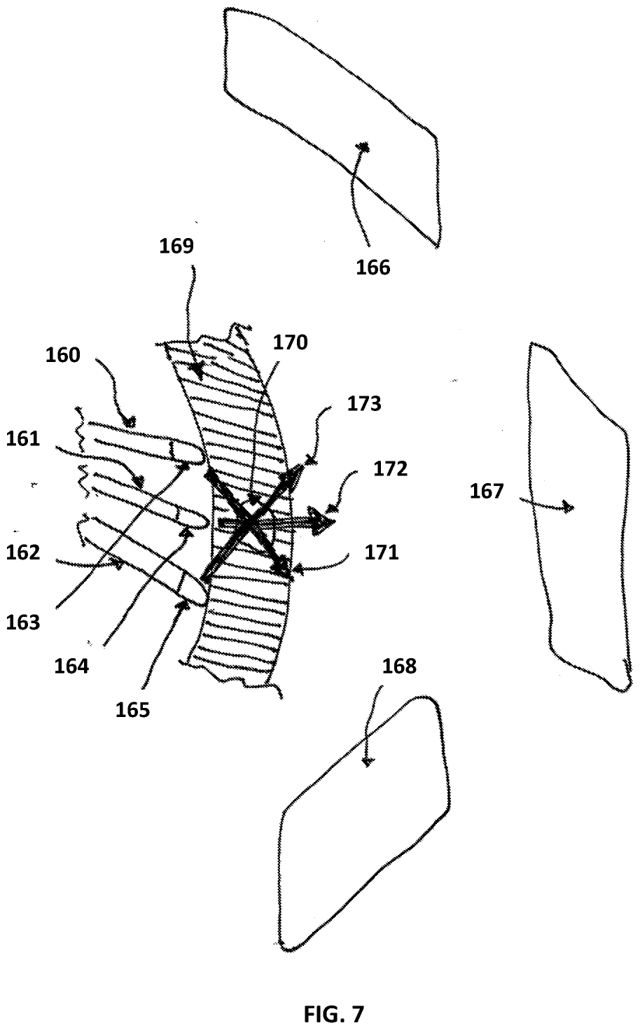

In certain embodiments, an ablation target tissue may be identified and targeted with the ablation catheter in different locations. A catheter may not be in a stable position and move incidentally. Or additional ablation may be desired to assure that a target tissue is fully ablated. FIG. 7 illustrates the distal aspect of an ablation catheter in different positions and using ablation vectors to target a specific tissue. In this illustration, the distal aspect of an ablation catheter is moved in sequential positions 160, 161, 162 with the ablation electrode initially in position 163, 164, and 165. Mapping of an arrhythmia and endocardium of the heart may have been performed using a 3D mapping system to create an electro-anatomic map. From that map, the location of the tissue 169 may be identified and further the target tissue for ablation 170 may be identified. Based on the 3D location of the target tissue 170 and the ablation electrode positions 163, 164, 165, ablation vectors 171, 172, 173 may be calculated respectively so that the ablation vector transects the target tissue. The illustrated series of multifunction patches 166, 167, 168 are part of a complete set of multifunction patches that substantially surround the potential ablation electrode positions. The complete set of multifunction patch locations may be used as vertices to create a triangulated mesh. Ablation vectors 171, 172, 173 may be projected to the triangulated mesh and appropriate ablation currents delivered between the ablation electrode and the multifunction patch array.

In certain embodiments, vector ablation may be applied according to an electro-anatomic map of the heart. The energy delivery to the heart may be calculated and displayed for physician review. Approximation of radiofrequency lesion creation may be performed using a combination of radiofrequency energy time, radiofrequency energy power, 3-dimensional location, contact force, approximation of resistive heating, and approximation of conductive heating.

In certain embodiments, respiration may be monitored by changes in the magnetic electrode sensors and chest impedance from the multifunction patch array and, particularly, the ECG electrode component. The heart rhythm may be tracked with each type of beat registered. Thus, the multifunction array may be used for tracking respiration and rhythm and the influence of respiration and rhythm on the heart's position.

In certain embodiments, an improved representation of the chest and heart may be derived from ultrasound, computed tomography, magnetic resonance imaging, or other imaging techniques. The resultant representation of anatomy is used to derive a finite element model (FEM) of the heart and chest. The FEM is a patient specific model and will have impedance is labeled for various tissues. Additionally, the model will use actual impedance data to further improve patient specificity of the model. The model will be registered to the electro-anatomic model of the heart. Since the heart, chest, and location of dispersion patches are included in the model, radiofrequency energy may be delivered in a patient specific manner to both a computational model of the heart and to the actual physical heart simultaneously. The finite element model may include resistive and conductive heating. As radiofrequency ablation is performed, accurate feedback may be given to a physician as to the efficacy of radiofrequency ablation and the expected ablation lesion development. A FEM of the heart and chest that is patient specific will improve radiofrequency ablation efficacy and safety.

### IV. ADDITIONAL FEATURES

In another aspect, the present disclosure provides an array of patches, wherein each patch comprises a 3D position sensor. In certain embodiments, each patch may also comprise a dispersion element (e.g., each patch may be a multifunction patch as described herein).

Certain benefits may be achieved using an array of such patches. For example, an important issue and cardiac mapping is instability of the reference system which unfortunately may cause map distortions. Those map distortions may have negative procedural effects including reducing procedural efficacy and reducing procedural safety. Thus, it is highly desirable to have a very stable reference system. Traditional reference systems utilize the table as the reference. However, that methodology often results in map distortions which have negative procedural effects. An array of patches, wherein each patch comprises a 3D position sensor, allows the patient to be used as the reference. More specifically, the array of locatable, and preferably multifunction, patches are used as the patient reference. A simple “central zero” reference is then the average position in XYZ coordinates of the multifunction patches. Yaw, pitch, and roll of the patient may be measured relative to the array of multifunction patches to maintain the correct coordinates system.

As another example, an important problem with current mapping systems is “impedance drift.” If a set of impedances is measured relative to the magnetic system and both remain stable, then impedance measurements and magnetic measurements remain correctly linked. However, if the impedance patches or impedance patch component of the multifunction patches have gradual impedance changes at the patch-patient skin interface, then the phenomenon is of impedance drift may occur. With this phenomenon, the impedance map is no longer appropriately linked to the magnetic map and a map distortion occurs. This type of phenomenon is highly undesirable.

The present disclosure provides a solution to the impedance drift problem. In particularly, the magnetic XYZ coordinates are associated with the derivative of the impedance X′Y′Z′ coordinates. The spatial derivative of impedance within the heart and the chest is not expected to change substantially within a procedure. Thus, by measuring the position of electrodes relative to an active mapping catheter that has both impedance and magnetic measurements, the derivative space will yield an accurate impedance map without being subjected to the impedance drift phenomenon. FIG. 3 illustrates the relationships between “Magnetic Space X,Y,Z”, “Impedance Space X′Y′Z′”, and “Derivative Impedance Space dX′dY′dZ′”. “Impedance drift” may occur between impedance space and magnetic space, however the relationship within the chest and the heart for derivative impedance space and magnetic space is expected to be “driftless.” When live magnetic and impedance data are added to the magnetic and derivative impedance space, then impedance space should remain accurate over time and produce a “Driftless Magnetic and Impedance Model.”

In certain embodiments, when two catheters in the heart that have the ability of both magnetic and impedance measurements, then active monitoring of the impedance gradient may be measured by the relative impedance distances in X′Y′Z′ and XYZ space.

In certain embodiments, it is highly desirable to have an integrated ultrasound system with a cardiac mapping system to appropriately identify cardiac anatomic structures and facilitate ablation of arrhythmias when they are anatomically challenging. An advantage of having a patient centered reference is that additional data may be obtained relative to the patient centered reference and be used to create a structural model of the heart relative to electrophysiologic characteristics of the heart. FIG. 4 illustrates a heart 40, in a long axis view. Ultrasound probe position 41 and 42 yield image slices 43, 44, 45, and 46. The ultrasound probe has an associated and calibrated magnetic positioning system with X, Y, and Z coordinates along with yaw, pitch, and roll such that the ultrasound. The ultrasound probe may be an external ultrasound transducer, a trans-esophageal ultrasound transducer, or an intracardiac ultrasound transducer. Ultrasound images may be combined from one or more ultrasound probes into the anatomic model. When multiple images are combined, ultrasound reflectance, ultrasound Doppler, and other ultrasound parameters may be projected accurately relative to the patient onto a 3D ultrasound model. The data may be gaited to cardiac rhythm and respiration to generate the most accurate structural model possible. When time is included as a function of gaiting, the ultrasound model is considered a 4D ultrasound model.

In another aspect, modifications may be performed of the ablation electrode and surrounding environment to improve ablation efficacy. These modifications may include making the ablation electrode more spherical. This will help minimize variations in contact force based on electrode to tissue angle and will also create a more homogeneous contact area of the ablation electrode versus tissue. This may improve consistency of the fraction of radiofrequency energy and a trading tissue relative to contact force and contact area. The surrounding environment may be modified by irrigating the ablation electrode with a less conductive ionic solution such as half-normal saline or dextrose-5 water. A lower ionic irritant solution will increase the resistivity of radiofrequency current into the blood pool and improve the fraction of current delivered into tissue.

### V. SPECIFIC EMBODIMENTS

In one aspect, the present disclosure provides a system for tissue ablation. The system comprises: (a) a catheter having at least one ablation element; (b) a plurality of multifunction patches, each multifunction patch having a 3D position sensor and a dispersion element, wherein the at least one ablation element is configured to deliver current to at least one of said plurality of multifunction patches, thereby creating an ablation vector. In certain embodiments, the tissue is cardiac tissue. In certain embodiments, at least one ablation element is a tip electrode and/or the energy is radiofrequency energy. In certain embodiments, the energy is direct current, alternating current, pulsed direct current, or any combination of these for electroporation ablation. In certain embodiments, the system further comprises at least one magnetic field emitter. In certain embodiments, the 3D position sensor comprises one or more magnetic field sensors. In certain embodiments, the plurality of multifunction patches comprises at least six multifunction patches. In certain embodiments, the plurality of multifunction patches are arranged in a pattern that substantially surrounds an ablation site. In some such embodiments, the pattern is a geometric pattern. In certain embodiments, the system further comprises a plurality of sensors operatively connected to the plurality of multifunction patches, the plurality of sensors configured to sense energy delivered to and/or from each respective multifunction patch.

In one aspect, the present disclosure provides a method for ablating mammalian tissue. The method comprises: (a) applying a plurality of dispersive patches to a patient, each of said plurality of dispersive patches having a 3D position sensor and a dispersion element; and (b) differentially directing an ablative current to said plurality of dispersive patches. In certain embodiments, the method further comprises obtaining position information from at least one of the plurality of dispersive patches. In certain embodiments, the method further comprises calculating at least one ablation vector based on the location information. In certain embodiments, the plurality of dispersive patches are applied in array surrounding an ablation site. In certain embodiments, the ablation site comprises cardiac tissue. In certain embodiments, the method further comprises ablation lesion modeling.

Certain methods and systems disclosed herein may be implemented in or include hardware, software, firmware or any combination of these. In certain embodiments, methods (or particular method steps) are implemented as computer software running on one or more processors. As such, these methods may be implemented in a single unit, or may be physically and functionally distributed between different units and processors. In such methods and systems, the processor may be any suitable processing device or set of processing devices such as, but not limited to, a microprocessor, a microcontroller-based platform, an integrated circuit, one or more field programmable gate arrays (FPGAs), and/or one or more application-specific integrated circuits (ASICs).

Certain methods and systems disclosed herein may include a memory configured to store one or more sets of instructions, such as software for operating the methods of the present disclosure. The memory may be volatile memory (e.g., RAM including non-volatile RAM, magnetic RAM, ferroelectric RAM, etc.), non-volatile memory (e.g., disk memory, FLASH memory, EPROMs, EEPROMs, memristor-based non-volatile solid-state memory, etc.), unalterable memory (e.g., EPROMs), read-only memory, and/or high-capacity storage devices (e.g., hard drives, solid state drives, etc.). In certain embodiments, the memory includes multiple kinds of memory, particularly volatile memory and non-volatile memory. In certain embodiments, the instructions reside completely, or at least partially, within any one or more of the memory, a computer readable medium, and/or within the processor during execution of the instructions.

The terms “non-transitory computer-readable medium” and “computer-readable medium” include a single medium or multiple media, such as a centralized or distributed database, and/or associated caches and servers that store one or more sets of instructions. Further, the terms “non-transitory computer-readable medium” and “computer-readable medium” include any tangible medium that is capable of storing, encoding or carrying a set of instructions for execution by a processor or that cause a system to perform any one or more of the methods or operations disclosed herein. As used herein, the term “computer readable medium” is expressly defined to include any type of computer readable storage device and/or storage disk and to exclude propagating signals.

In this application, the use of the disjunctive is intended to include the conjunctive. The use of definite or indefinite articles is not intended to indicate cardinality. In particular, a reference to “the” object or “a” and “an” object is intended to denote also one of a possible plurality of such objects. Further, the conjunction “or” may be used to convey features that are simultaneously present instead of mutually exclusive alternatives. In other words, the conjunction “or” should be understood to include “and/or”. The terms “includes,” “including,” and “include” are inclusive and have the same scope as “comprises,” “comprising,” and “comprise” respectively.

While certain of the preferred embodiments of the present invention have been described and specifically exemplified above, it is not intended that the invention be limited so such embodiments. Various modifications may be made thereto without departing from the scope of the present invention.

What is claimed is:

1. A multifunction patch having a 3D position sensor and a dispersion element.

2. The multifunction patch of claim 1, further comprising an ECG electrode element.

3. The multifunction patch of claim 2, wherein the dispersion element surrounds the ECG electrode element or is adjacent to the ECG electrode element.

4. The multifunction patch of claim 1, wherein the dispersion element comprises an electrically conductive material, preferably an electrically conductive gel.

5. The multifunction patch of claim 1, wherein the 3D position sensor comprises one or more magnetic field sensors.

6. The multifunction patch of claim 1, wherein the 3D position sensor is a tri-axial sensor.

7. The multifunction patch of claim 1, further comprising an adhesive element suitable for adhering the patch to human skin.

8. A system comprising a plurality of patches, each patch having an electrode; wherein said plurality of patches are arranged as an array substantially surrounding an ablation site.

9. The system of claim 8, wherein the patches are multifunction patches.

10. The system of claim 9, wherein each patch further comprises a 3D position sensor.

11. The system of claim 10, further comprising a magnetic field emitter, wherein the 3D position sensor comprises one or more magnetic field sensors.

12. The system of claim 11, further comprising a processor for receiving and processing a signal generated by the 3D position sensor and the processor is preferably configured to determine the position coordinates of at least one of the plurality of multifunction patches.

13. A method for ablating mammalian tissue, the method comprising:
applying an ablative current between one or more ablation electrodes and one or more dispersion elements of a dispersion patch array, wherein the current is differentially directed to one or more dispersion elements according to a desired ablation vector.

14. The method of claim 13, wherein the ablative current comprises radiofrequency energy.

15. The method of claim 13, wherein the ablative current comprises an electrical current for electroporation.

16. The method of claim 13, wherein the ablation vector is a surface normal vector that is perpendicular to the tissue.

17. The method of claim 13, wherein the ablation vector is aligned to a contact force vector.

18. The method of claim 13, wherein the ablative current comprises independent currents.

19. The method of claim 13, wherein the ablative current comprises a single current.

20. The method of claim 19, wherein two or more dispersion patches of the dispersion patch array are grounded together.

21. The method of claim 13, further comprising the step of determining the position coordinates of at least one dispersion patch of the dispersion patch array, preferably using triangulation and estimation from multiple impedance measurements relative to other electrodes with known locations and/or calculated locations.

22. The method of claim 13, wherein each dispersion patch of the dispersion patch array comprises a 3D position sensor.

23. The method of claim 22, further comprising the step of determining the position coordinates of at least one dispersion patch of the dispersion patch array based on a signal provided by the 3D position sensor.

24. The method of claim 13, further comprising the step of updating the desired ablation vector to target selected tissue in three-dimensional space.
